(19) United States
(12) Patent Application Publication
(10) Pub. No.: US 2016/0263852 A1
(43) Pub. Date: Sep. 15, 2016

(54) GLOVE MANUFACTURING METHODS AND ASSEMBLIES

(71) Applicant: BRIDGE MEDICAL PRODUCTS, INC., Bellevue, WA (US)

(72) Inventor: H. Peter Koenen Myers, Oklahoma City, OK (US)

(21) Appl. No.: 15/030,581

(22) PCT Filed: Oct. 20, 2014
(86) PCT No.: PCT/US2014/061408

§ 371 Date: Apr. 19, 2016

Related U.S. Application Data
(60) Provisional application No. 61/893,406, filed on Oct. 21, 2013.

(51) Int. Cl. B29D 99/00; B29C 41/42; A41D 19/00; A61B 42/10; A61B 90/30; A41D 19/04; B29C 41/14; B29C 41/40

(52) U.S. Cl. CPC B29D 99/0067; B29C 41/14; B29C 41/42; B29C 41/40; A61B 42/10; A61B 90/30; A41D 19/04; A41D 19/0082; B29K 2019/00

(57) ABSTRACT

Glove manufacturing methods can include a hand mandrel and a tube mandrel. The method can include dipping the hand mandrel and the tube mandrel while the mandrels are positioned apart from each other. The method can include bringing the hand mandrel in close proximity to the tube mandrel to enable creating a glove with an attached tube. The tube can be used to hold at least one optical fiber to enable light transmission distally along a finger. In several embodiments, the tube can be used for suction, irrigation, cautery, ultrasound, and/or sensing instruments. Gloves, including surgical gloves, can be made from many different materials including latex, nitrile, neoprene, cloth, leather, and/or a combination thereof.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. §1.57.

### BACKGROUND OF THE INVENTION

1. Field of the Invention

Various embodiments disclosed herein relate to gloves. Certain embodiments relate to methods and assemblies that can be used to manufacture gloves.

2. Description of the Related Art

Gloves can be used in many activities, procedures, and situations. Gloves can be made from many materials including latex, neoprene, nitrile, cloth, and leather. Gloves can protect a person's hands. In medical procedures and surgeries specifically, gloves can protect medical professionals and patients from harmful fluid contact. Fluid contact can pose a risk to medical professionals and patients. For example, latex gloves can prevent blood transfer from the patient to the medical professional. Blood transfer can transmit dangerous conditions such as human immunodeficiency virus (“HIV”) and hepatitis.

Medical professionals often need artificial light to see inside of a patient's body during many surgeries. Current sources of artificial light include lights worn on the head. There exists a need for a means to manufacture a glove that is compatible with a hand-mounted light emitter.

### SUMMARY OF THE INVENTION

A method is disclosed whereby a hand and tube mandrel system is rotated by a first gear, which then further rotates a first shaft coupled to a glove mandrel with a groove shaped along a finger of the glove mandrel. The system is further rotated with a second gear, which then further rotates a second shaft coupled to a tube mandrel shaped to match the groove of the glove mandrel. Next, the system is further rotated with a third shaft, wherein the third shaft is coupled to a base, wherein the base is coupled to the first gear and the second gear. In some embodiments the system is dipped into a dipping container, which contains material capable of adhering to the glove mandrel and the tube mandrel.

Another embodiment relates to a system including a shaft; a base connected to the shaft, which is operable to the move the base; a hand mandrel with one or more grooves, coupled to the base; and one or more tube mandrels coupled to the base, where the one or more tube mandrels corresponds to at least one of the one or more grooves.

Another embodiment relates to a system which includes a glove with one or more digits and one or more tubes, coupled to the exterior of at least one of the one or more digits.

### BRIEF DESCRIPTION OF THE DRAWINGS

FIG. 1 is a block diagram illustrating a perspective view of a hand mandrel and a tube mandrel;

FIG. 2 is a block illustrating a perspective view of a mandrel assembly and a dipping container;

FIG. 3 is a block diagram illustrating a side view of a mandrel assembly at least partially inside of a dipping container;

FIG. 4A is a block diagram illustrating a perspective view of a joining position;

FIG. 4B is a block diagram illustrating a side view of a joining position;

FIG. 5A is a block diagram illustrating a side view of a joining position;

FIG. 5B is a block diagram illustrating a perspective view of a joining position;

FIG. 6 is a block diagram illustrating a side view of a hand mandrel;

FIG. 7 is a block diagram illustrating a front view of a hand mandrel;

FIG. 8 is a block diagram illustrating a perspective view of a horizontal mandrel assembly;

FIG. 9 is a block diagram illustrating a side view of a tube mandrel removal system;

FIG. 10 is a block diagram illustrating a perspective view of a tube mandrel removal system;

FIGS. 11A-11B are block diagrams illustrating perspective views of a mandrel assembly that includes a removal system;

FIG. 12 is a block diagram illustrating a perspective view of a mandrel assembly; and

FIG. 13 is a block diagram illustrating a perspective view of an embodiment that uses several mandrel assemblies.

### DETAILED DESCRIPTION OF THE PREFERRED EMBODIMENT

Features, aspects, and advantages are described below with reference to the drawings, which are intended to illustrate but not to limit the invention. In the drawings, like reference characters denote corresponding features consistently throughout similar embodiments.

Although certain embodiments and examples are disclosed herein, inventive subject matter extends beyond the examples in the specifically disclosed embodiments to other alternative embodiments and/or uses, and to modifications and equivalents thereof. Thus, the scope of claims is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. No feature, benefit, advantage, structure, or step disclosed herein is essential or indispensable. Many alternatives may be within the scope of the inventions described herein.

The drawings illustrate certain embodiments and are not intended to be limiting. The drawings can be semi-diagrammatic and not to scale. For clarity of presentation and discussion, some portions of and/or dimensions in the drawings are shown greatly exaggerated.

For expository purposes, the term “horizontal” as used herein is defined as a plane parallel to the plane or surface of the floor of the area in which the device being described is used or the method being described is performed, regardless of its orientation. The term “floor” can be interchanged with the term “ground.” The term “vertical” refers to a direction perpendicular to the horizontal as just defined. Terms such as “above,” “below,” “bottom,” “top,” “side,” “higher,” “lower,” “upper,” “over,” and “under,” are defined with respect to the horizontal plane unless otherwise indicated.

Several embodiments include at least one hand mandrel and a least one tube mandrel. As used herein, a hand mandrel can be a hand-shaped former that can be coated with a coagulant (e.g., calcium nitrate) and dipped into a container of liquid latex. The latex can coat the hand mandrel with a thin film of latex. A tube mandrel can be a former that is shaped to create a channel on a glove. The channel can have a circular cross section, a rectangular cross section, an oval cross section, or any cross section suitable for optical fibers. A light transmitting device can be placed inside of the channel. Optical fibers are one type of light transmitting device. The channel can be used to conduct/guide optical fibers to the fingertips of gloves. In several embodiments, the channel can be used for suction, irrigation, cautery, ultrasound, and/or sensing devices. For example, fluid can be removed from a wound via the channel. Fluid can be introduced into a surgical site via the channel. In some embodiments, the channel can carry electrocautery devices and/or ultrasound devices. Sensing systems can be at least partially located in the channel. In several other embodiments, the channel can be used to carry/guide other devices including: endoscopic fibers, ultrasound devices, LEDs, laser lights and/or laser devices, suction and irrigation devices, camera devices and/or video devices, or any device capable of sampling, metering, or sensing (e.g., pH meters/sensing).

The light transmitting device (e.g., a fiber optic bundle) can extend from a wrist of a medical professional (or the wrist portion 74 of a glove) to at least one fingertip portion 78 (e.g., of the medical professional and/or of a glove). Some embodiments include endoscopic fiber optic bundles. In several embodiments, a channel that extends from a wrist of a medical professional (or the wrist portion 74 of a glove) to at least one fingertip portion 78 can be used for suction, irrigation, cautery, ultrasound, and/or sensing devices.

Several method embodiments include creating a surgical glove with a tube configured to hold a light transmitting device. The method can include using the hand mandrel and the tube mandrel to manufacture a surgical glove with the tube. Some methods include placing optical fibers inside of the tube and conducting light from the wrist area to a fingertip area. As used herein, a fingertip area can include the area of a fingernail (e.g., immediately above a fingernail).

Gloves, including surgical gloves, can be made from many different materials including latex, vinyl, nitrile, neoprene, cloth, leather, and/or a combination thereof. Some manufacturing methods include dipping at least one mandrel in latex, vinyl, nitrile, neoprene, and/or a combination thereof to form a thin layer of material. The thin layer can become the glove. Or in other manufacturing methods including a tube, the thin layer may be sewn/woven together, for example, a cloth or leather glove.

The hand mandrel and the tube mandrel can be dipped separately in latex, removed from the latex bath, and then brought together for a curing process such that a portion of the latex that coats the hand mandrel touches a portion of the latex that coats the tube mandrel. For example, in a cuff beading process, the hand mandrel may turn independently of the tube mandrel and then be cured. After the curing process, the tube mandrel can be withdrawn from the tube. In some embodiments, the tube is a conduit for a light transmitting device. In other embodiments, the tube and glove mandrel are in close proximity, allowing the combined unit after the curing process to be blown off with a gas such as air as a single unit. For example, in one embodiment, the tube and glove mandrel may have no moving parts, thereby allowing the final cured product to exist as a single unit.

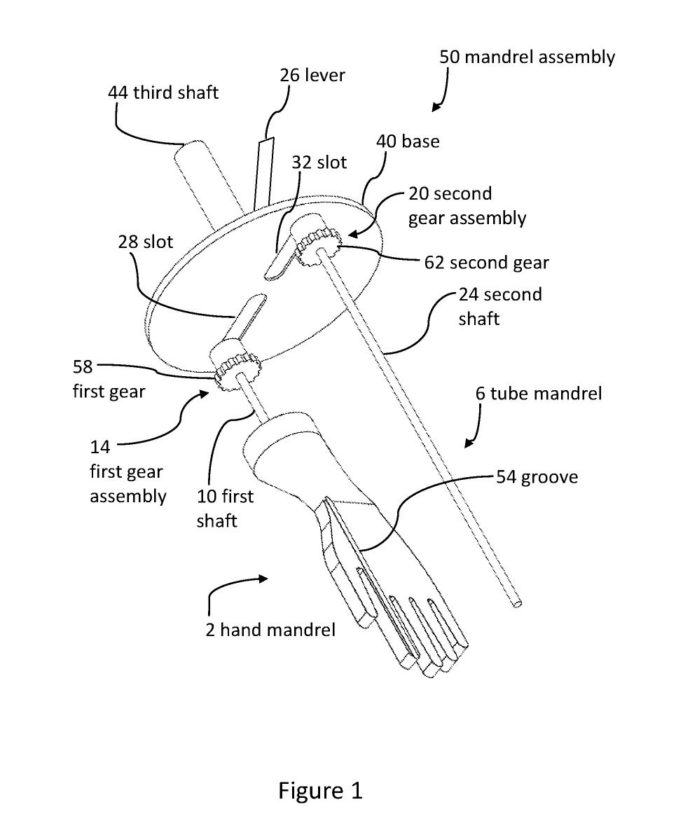

FIG. 1 illustrates a perspective view of a hand mandrel 2 and a tube mandrel 6. The tube mandrel 6 can be a long, metal rod with a diameter configured for the target diameter of the tube. The tube mandrel 6 can include a screw mechanism configured to pull the tube mandrel 6 out of a glove (e.g., retract the tube mandrel 6 in a proximal direction). A first shaft 10 can be configured to rotate. This rotatable first shaft 10 can be coupled to a first gear assembly 14. The first gear assembly 14 can include a motor configured to rotate the first shaft 10. When the first shaft 10 is coupled to the hand mandrel 2, the first gear assembly 14 can rotate the hand mandrel 2.

A second gear assembly 20 can be configured to rotate a second shaft 24 to rotate the tube mandrel 6. A lever 26 can be used to move the tube mandrel 6 in close proximity to the hand mandrel 2 in order to form a tube attached to a glove. The first gear assembly 14 and the second gear assembly 20 can slide in slots 28, 32 when the lever 26 is activated. The rotation of the hand mandrel 2 and/or the tube mandrel 6 can be monitored to enable the system to enable a suitable rotational orientation between the hand mandrel 2 and the tube mandrel 6 when the tube mandrel 6 is brought in close proximity to the hand mandrel 2.

The hand mandrel 2 and the tube mandrel 6 can be coupled by a base 40, which can be a cylinder that is configured to rotate. A third shaft 44 can be coupled to the base 40 such that rotating the third shaft 44 can rotate the base 40, and thereby, can rotate the hand mandrel 2 and the tube mandrel 6 independently. A motor can be used to rotate the third shaft 44. Rotating the third shaft 44 can enable the hand mandrel 2 and the tube mandrel 6 to rotate together (e.g., during the curing process). The mandrel assembly 50 can be utilized in a continuous production line.

In various embodiments, the hand mandrel 2 and the tube mandrel 6 can be coupled to the base 40 directly, without the use of first gear 58, first gear assembly 14, second gear 62, and second gear assembly 20. The hand mandrel 2 and the tube mandrel 6 may be in close proximity to one another, connected only to the base 40. In such embodiments, the third shaft 44 may enable the hand mandrel 2 and the tube mandrel 6 to rotate together (e.g., during the curing process). This mandrel assembly without moving parts (first gear 58, first gear assembly 14, second gear 62, and second gear assembly 20) may allow the mandrel assembly to be utilized in a continuous production, whereby the completed unit after the curing process may be “blown off”, known to those skilled in the art. For example, third shaft 44 may have a conduit coupled traveling through to the hand mandrel 2 and the tube mandrel 6. Such a conduit may enable a pressurized air component coupled to the third shaft 44 to blow off the completed unit after the curing process (e.g., through one or more holes in the hand mandrel 2 and tube mandrel 6, allowing an air flow through those one or more holes from the pressurized air component). In another embodiment, pressurized air may be blown down on the exterior surface of a cured single glove and tube unit. In other embodiments, the pressurized air component may be connected to the hand mandrel 2 and the tube mandrel 6 via the base 40. Various other configurations of coupling a pressurized air component (or any component allowing air, liquid or gaseous transfer to the hand mandrel 2 and the tube mandrel 6) to mandrel assembly 50 are possible.

The hand mandrel 2 can have a groove 54 configured for the tube mandrel 6 to sit at least partially within the groove 54. In some embodiments, the groove 54 extends from a wrist area of the glove to a fingertip of the glove or to a fingernail portion 78 of the glove. Some embodiments include a flat area in the area shown as the groove 54. Some embodiments include a standoff in the area shown as the groove 54 to allow space (e.g., clearance) between the rest of the hand mandrel 2 and the tube mandrel 6. This configuration can leave a free section of tube unconnected to the glove formed by the hand mandrel 2.

The hand mandrel 2 can have at its base a first gear 58. The tube mandrel 6 can have at its base a second gear 62. The first gear 58 and the second gear 62 can have the same number of teeth (e.g., the gear ratio can be 1:1). In some embodiments, a chain, a strap, or a belt mechanically couples the first gear 58 to the second gear 62 such that turning one gear causes the other gear to turn via the chain, strap, or belt.

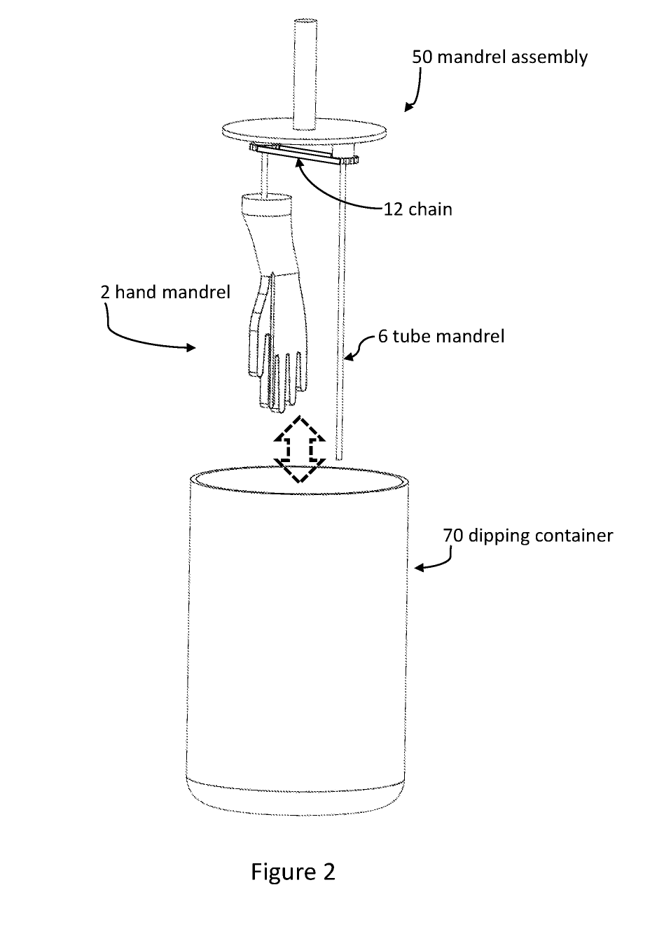

FIG. 2 illustrates a perspective view of a mandrel assembly 50 and a dipping container 70. A chain 12 is illustrated in FIG. 2, but the chain 12 is hidden in many figures in the interest of increasing the clarity of other items.

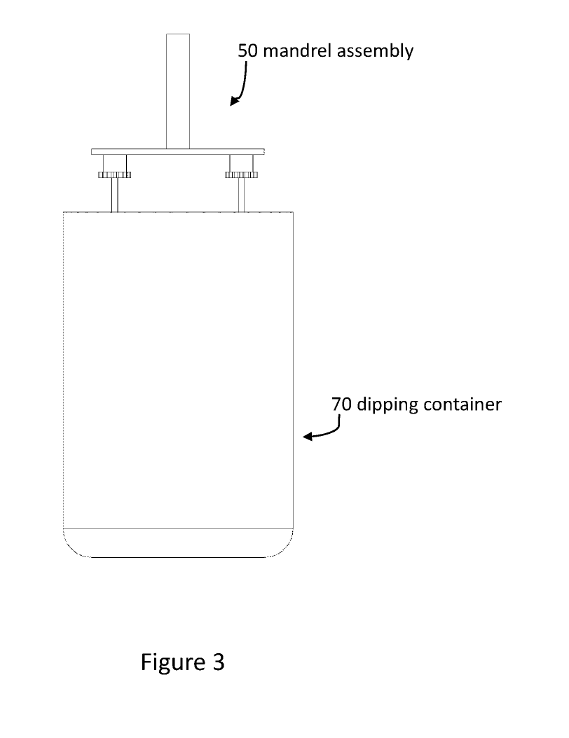

FIG. 3 illustrates a side view of a mandrel assembly 50 at least partially inside of a dipping container 70. Referring now to FIGS. 2 and 3, the dipping container 70 can hold a material used to make a glove. In some embodiments, the dipping container 70 holds latex, which can be natural or synthetic. The hand mandrel 2 and the tube mandrel 6 can be lowered into the dipping container 70. In some embodiments, the dipping container 70 is raised to immerse the hand mandrel 2 and the tube mandrel 6 in the dipping container 70. The hand mandrel 2 and the tube mandrel 6 can be dipped separately (e.g., when the hand mandrel 2 and the tube mandrel 6 are not touching). In some embodiments, the hand mandrel 2 and the tube mandrel 6 are dipped in the latex when the hand mandrel 2 and the tube mandrel 6 are at least 0.5 inches apart and/or less than 5 feet apart; at least 1 inch apart and/or less than 3 feet apart; or at least 3 inches apart and/or less than 2 feet apart.

A dashed arrow in FIG. 2 illustrates how the hand mandrel 2 and the tube mandrel 6 can be dipped in the dipping container 70 and then removed from the dipping container 70. The dipping can occur while the hand mandrel 2 and the tube mandrel 6 are oriented vertically; oriented within +/−10 degrees of vertical; or oriented within +/−25 degrees of vertical.

Referring to FIGS. 2 and 3, various other embodiments of mandrel assembly 50 are possible. For example, the hand mandrel 2 and the tube mandrel 6 may be in close proximity to one another. In other embodiments, no moving parts may exist such that the hand mandrel 2 and tube mandrel 6 are directly connected to some fixed object of mandrel assembly 50, for example, the base 40. In various other embodiments, one or more tube mandrels 6 may be in close proximity, or even coupled/connected to the fingers of hand mandrel 2. In some embodiments, several tube mandrels 6 may be connected to one finger of the hand mandrel 2. In such embodiments, after the mandrel assembly 50 is lowered into the dipping container 70 (e.g., integrally dipped) and the curing process is completed, the complete unit of glove and tube(s) may be stripped or blown off of the hand mandrel 2 and tube mandrel(s) 6.

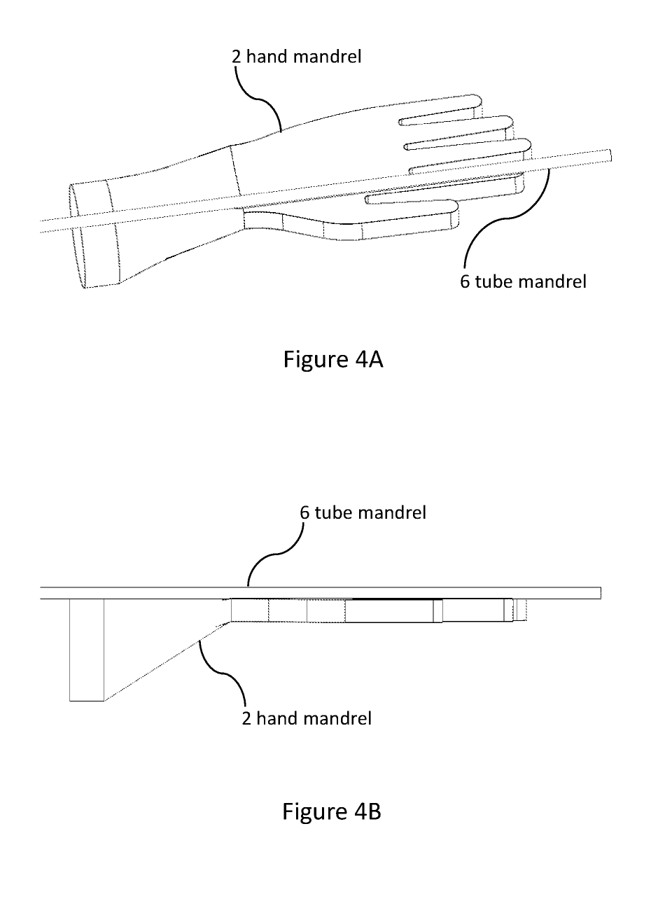

FIG. 4A illustrates a perspective view of a joining position. FIG. 4B illustrates a side view of a joining position. The tube mandrel 6 and the hand mandrel 2 are oriented horizontally in FIG. 4B. Referring now to FIGS. 4A and 4B, once the hand mandrel 2 and the tube mandrel 6 have been dipped separately, the tube mandrel 6 and/or the hand mandrel 2 can be moved into a joining position. The joining position can be configured to enable the material that coats the tube mandrel 6 to bond with the material that coats the hand mandrel 2. For example, a portion of the material that coats the tube mandrel 6 can bond with a portion of the material that coats the hand mandrel 2 in the groove 54 (shown in FIG. 1). The joining position can be when a portion of the material that coats the tube mandrel 6 touches a portion of the material that coats the hand mandrel 2. In the joining position, at least a portion of the tube mandrel 6 can be in proximity to at least a portion of the hand mandrel 2.

The hand mandrel 2 and the tube mandrel 6 can be locked in position (e.g., locked in place relative to each other) when they are in the joining position. The hand mandrel 2 and the tube mandrel 6 can be rotated together during the curing process. The hand mandrel 2 and the tube mandrel 6 can be oriented horizontally (e.g., as they are rotated) during the curing process. The curing can occur while the hand mandrel 2 and the tube mandrel 6 are oriented horizontally; oriented within +/−10 degrees of horizontal; or oriented within +/−25 degrees of horizontal.

Referring to FIGS. 4A-4B, various embodiments of the mandrel assembly 50 described above allow the hand mandrel 2 and the tube mandrel(s) 6 to be produced in close proximity of one another or further apart with gear assemblies as depicted in FIG. 1. After the curing process, the hand mandrel 2 and the tube mandrel(s) 6 may be joined in various methods known to those skilled in the art, including but not limited to, taping, stapling, sewing, gluing, VELCRO®, epoxy, strapping methods, and/or light or radiowave curing. In various embodiments, any glove dipped or sewn may have one or more tubes attached to the globe using any of the various methods described herein. For example, in one embodiment, VELCRO® may attach to one side of the glove (which may be, for example latex dipped or sewn); and the opposite VELCRO® piece may be attached to the tube. In other embodiments, the hand mandrel 2 and the tube mandrel(s) 6 may already be joined together if they were integrally dipped in a mandrel assembly 50 allowing such a curing process.

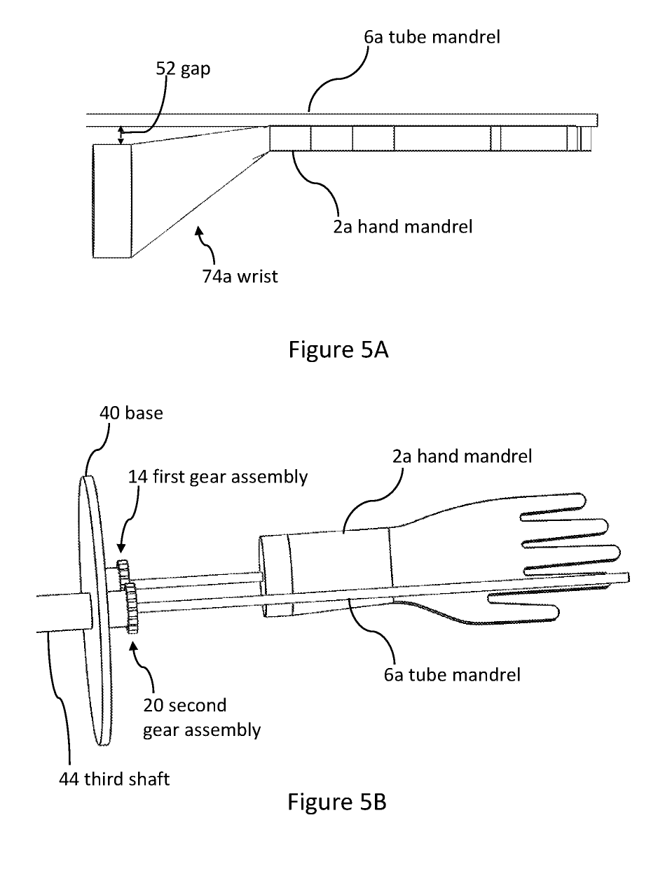

FIG. 5A illustrates a side view of a joining position. The tube mandrel 6a and the hand mandrel 2a are oriented horizontally in FIG. 5A. FIG. 5B illustrates a perspective view of the joining position. Referring now to FIGS. 5A and 5B, once the hand mandrel 2a and the tube mandrel 6a have been dipped separately, the tube mandrel 6a and/or the hand mandrel 2a can be moved into the joining position. The joining position can be configured to enable the material that coats the tube mandrel 6a to bond with the material that coats the hand mandrel 2a. While in the joining position, there can be a gap 52 between at least a portion of the wrist 74a of the hand mandrel 2a and at least a portion of the tube mandrel 6a. This gap 52 can be configured to enable a proximal portion of the tube formed by the tube mandrel 6a to not be directly bonded to the hand mandrel 2a. Thus, the system can form a glove with a tube that includes a free proximal end (e.g., that is at least 0.5 inches, at least 2 inches, at least 4 inches). The free proximal end can be coupled to the glove by a more distal portion of the tube formed by the tube mandrel 6a.

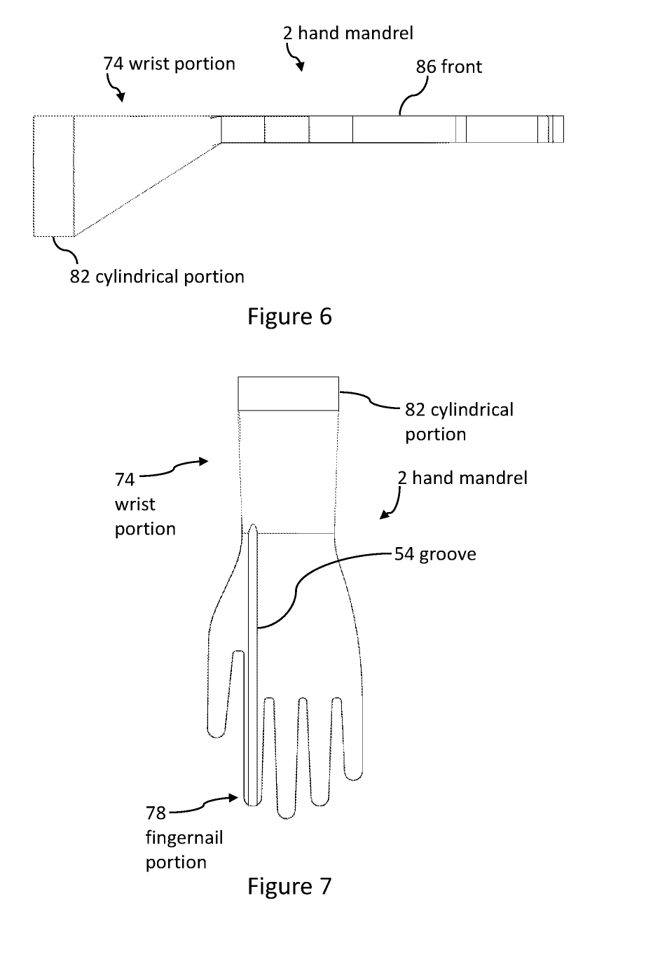

FIG. 6 illustrates a side view of the hand mandrel 2. The hand mandrel 2 can be shaped like a human's hand. FIG. 7 illustrates a front view of the hand mandrel 2. Referring now to FIGS. 6 and 7, the hand mandrel 2 can include a cylindrical portion 82 configured to enable a brush to form a beaded cuff on the proximal end of the glove. In some embodiments, the cylindrical portion is lower than the front 86 of the hand mandrel 2 to avoid the wrist portion 74 of the hand mandrel 2 getting in the way of the tube mandrel 6. In some embodiments, the wrist portion 74 does not extend past the front 86 of the finger portion of the hand mandrel 2.

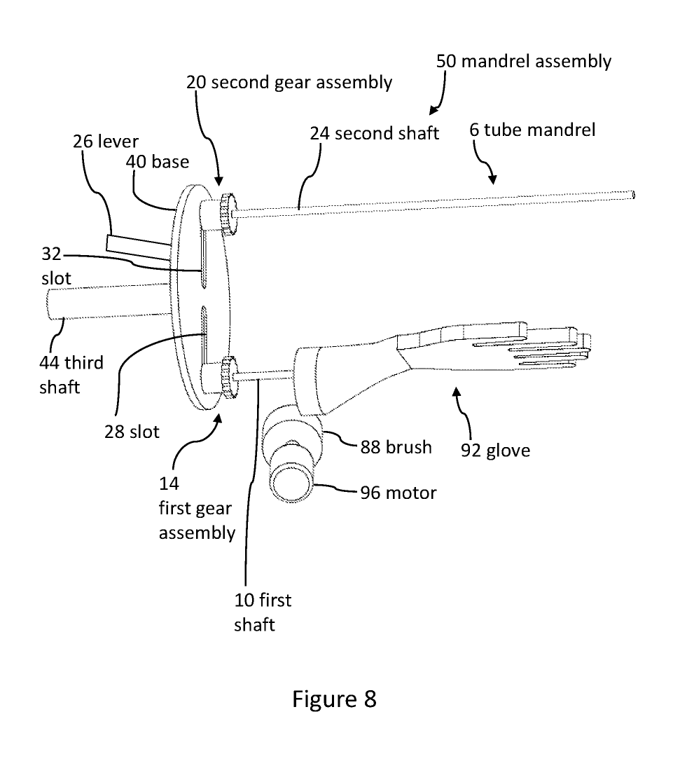

FIG. 8 illustrates a perspective view of a horizontal mandrel assembly 50 as the tube mandrel 6 and the hand mandrel 2 are rotated separately. A brush 88 rotates to form a cuff on the proximal end of a glove 92. The glove 92 covers the hand mandrel 2 (not shown). The brush 88 can be coupled to a motor 96 configured to rotate the brush 88.

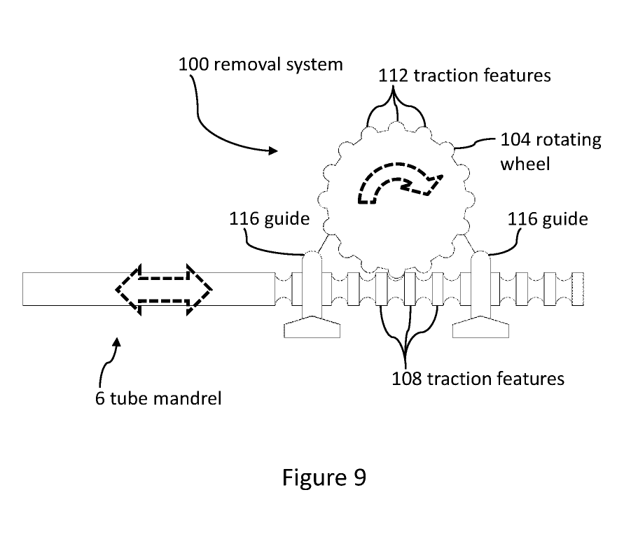

FIG. 9 illustrates a side view of a tube mandrel 6 removal system 100. In several embodiments, once the curing is done, the tube mandrel 6 can be removed by the removal system 100, which may be a screw mechanism, hydraulic system, or one or more levers. The removal system 100 can be located to the side of the tube mandrel 6.

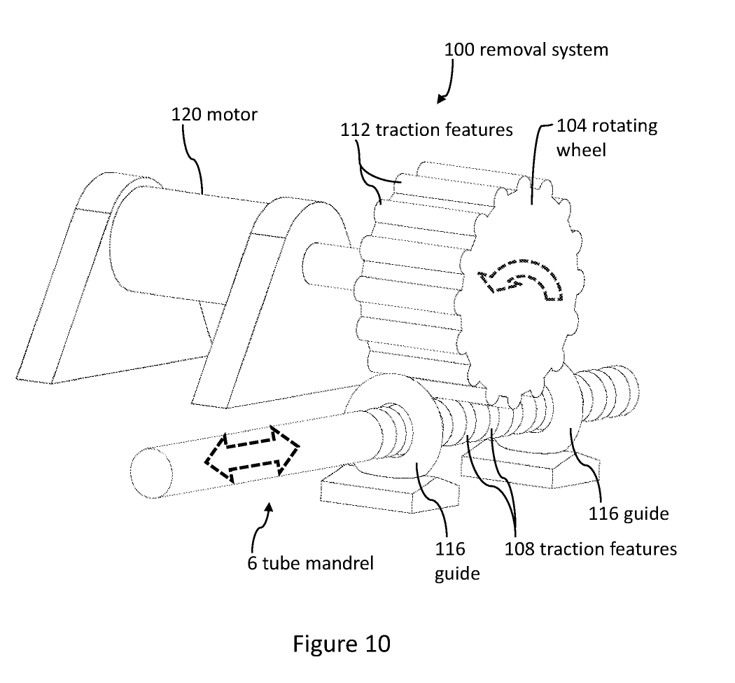

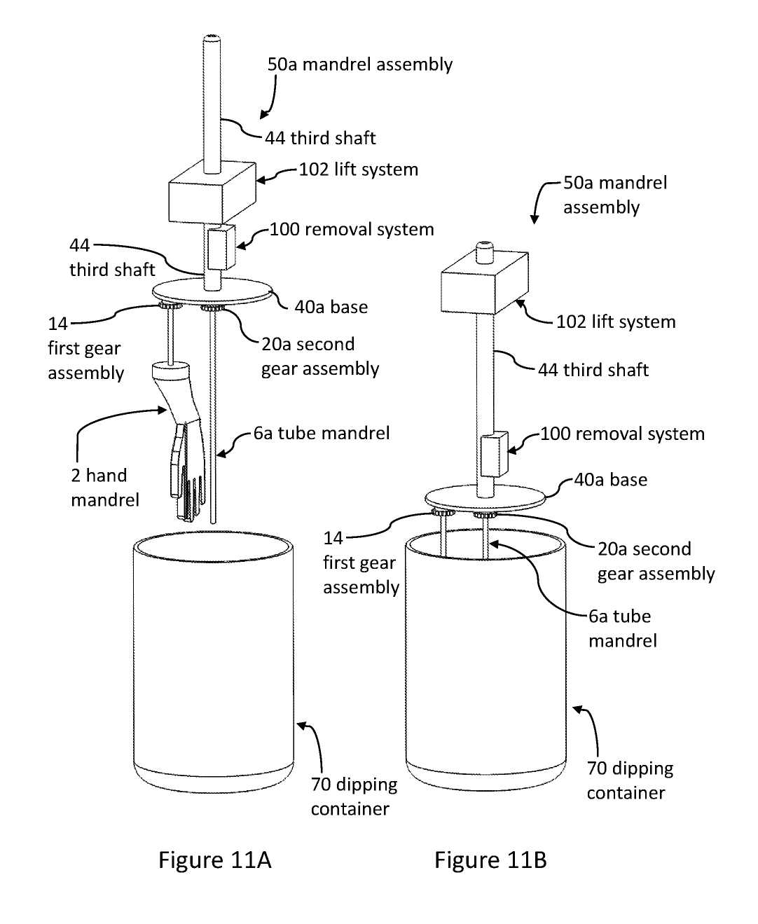

FIG. 10 illustrates a perspective view of the tube mandrel 6 removal system 100. Referring now to FIGS. 9 and 10, the removal system 100 can include a rotating wheel 104 configured to engage traction features 108 on the tube mandrel 6. The traction features 108 can be gear teeth, indentations, and/or protrusions. The rotating wheel 104 can also include traction features 112, which can be gear teeth, indentations, and/or protrusions. Not all of the traction features 108, 112 are labeled in FIGS. 9 and 10. The removal system 100 can include guides 116 (e.g., hoops, holes, channels, rings) through which the tube mandrel 6 passes. One guide 116 can be located on each side of the contact area between the rotating wheel 104 and the tube mandrel 6. A hole through the base 40a (shown in FIG. 11A) can serve as a guide 116. A hole in the base 40a can be a third guide.

The rotation of the rotating wheel 104 (e.g., a gear) can cause the tube mandrel 6 to move distally or proximally (depending on the direction of the rotation). As a result, the removal system 100 can retract the tube mandrel 6 out of a glove and can extend or return the tube mandrel 6 to a starting position (e.g., a dipping position). A shaft can couple a motor 120 to the rotating wheel 104 to enable the motor 120 to rotate the rotating wheel 104.

Once the tube mandrel 6 has been removed, the glove (e.g. a latex glove) can be stripped off the hand mandrel 2. The tube mandrel 6 can be screwed or moved back into a starting position and separated from the hand mandrel 2 for a subsequent manufacturing round (e.g., a next round of dipping). The mandrel assembly 50 (shown in FIG. 1) can be used during many manufacturing cycles to create many gloves. In other embodiments, rather than stripping, the glove and tube may be produced in one manufacturing round (e.g.., one round of dipping), allowing the combined unit to be blown off, for example, by a connected pressurized air unit.

FIGS. 11 A and 11 B illustrate perspective views of a mandrel assembly 50a that includes the removal system 100 illustrated in FIGS. 9 and 10. FIG. 11 A shows a configuration where the tube mandrel 6a and the hand mandrel 2 are located outside of the dipping container 70. The tube mandrel 6a and the hand mandrel 2 can be located outside of the dipping container 70 before dipping and/or after dipping. FIG. 11B shows the tube mandrel 6a and the hand mandrel 2 located inside of the dipping container 70 during a dipping (e.g., coating) process.

Referring now to FIGS. 11 A and 11 B, a lift system 102 can move the mandrel assembly 50a and/or a portion of the mandrel assembly 50a into and out of the dipping container. In some embodiments, the lift system 102 is configured to lower and raise the base 40a, the third shaft 44, and/or the mandrel assembly 50a. The third shaft 44 can slide up and down within the lift system 102.

The tube mandrel 6a can be coaxial with the third shaft to enable the removal system 100 to move at least a portion of the tube mandrel 6a into and out of the third shaft 44. The second gear assembly 20a can also be coaxial with the tube mandrel 6a and/or the third shaft 44. The guides 116 shown in FIG. 10 can be formed by a hole in the base 40a and/or by the third shaft 44.

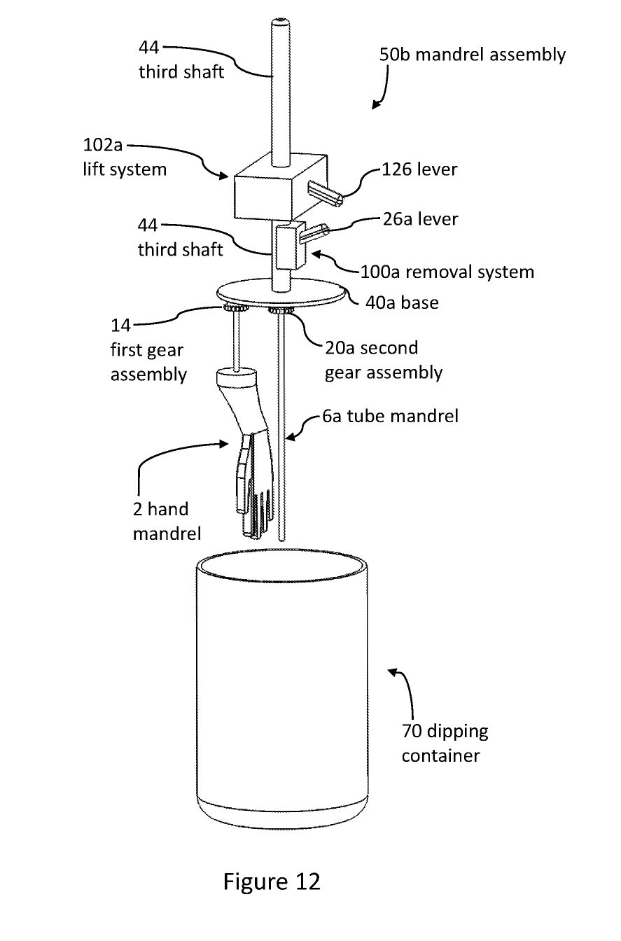

FIG. 12 illustrates a perspective view of a mandrel assembly 50b that is similar to the mandrel assembly 50a illustrated in FIGS. 11 A and 11 B. The lift system 102a can include a lever 126. Moving the lever 126 can raise and/or lower the mandrel assembly 50b relative to the lift system 102a.

The removal system 100a can also include a lever 26a. The lever 26a can be used to move the tube mandrel 6a in close proximity to the hand mandrel 2 in order to form a tube attached to a glove. The lever 26a can also be used to retract and/or extend the tube mandrel 6a. In some embodiments, moving the lever 26a to a first position causes the tube mandrel 6a to move in close proximity to the hand mandrel 2 such that material that coats the tube mandrel 6a contacts (e.g., bonds with) material that coats the hand mandrel 2. Moving the lever 26a to a second position can cause the tube mandrel 6a to retract (e.g., move proximally) relative to the hand mandrel 2.

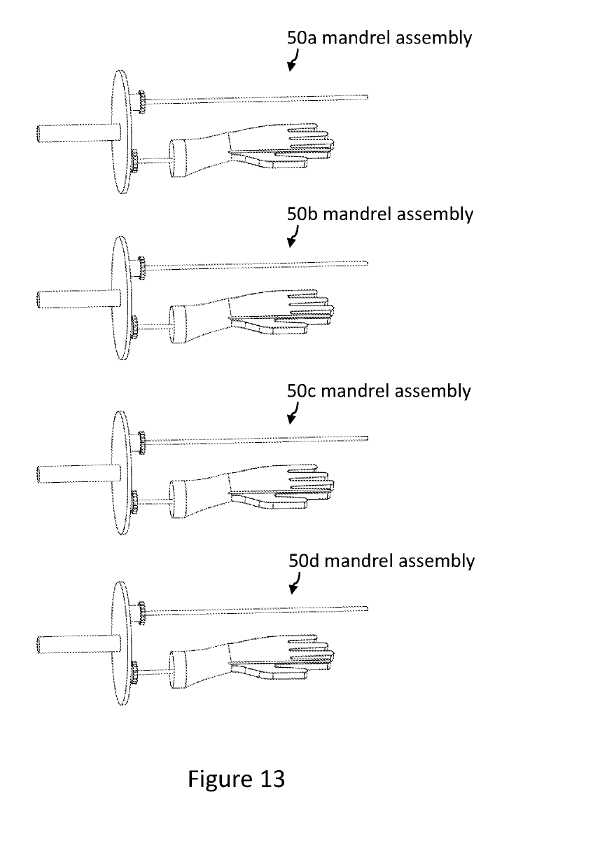

Many different methods of making a glove can use the hand mandrel 2 and the tube mandrel 6. Many embodiments only include a subset of the steps described herein. The steps can be performed in different orders than the orders explained through various embodiments. FIG. 13 illustrates a perspective view of an embodiment that uses many different mandrel assemblies 50a, 50b, 50c, 50d to create multiple gloves through a batch process, which can be automated or semi-automated to enable high-volume manufacturing.

In several embodiments, a hand mandrel includes a groove that acts as a negative to the tube mandrel positive. The tube mandrel and hand mandrel can be dipped separately. The hand mandrel can turn (e.g., rotate) to create a cuff bead via a brushing tool. The tube mandrel can turn to reduce variations in latex thickness (e.g., to keep the latex on the mandrel evenly). A lever mechanism can be used to bring the tube mandrel into a position proximate to the hand mandrel (e.g., a glove form). In some embodiments, once this has been accomplished, the individual rotation of the hand mandrel and the tube mandrel stops, and the hand mandrel and tube mandrel rotate as one unit through the curing process, through at least a portion of the curing process, through at least 65% of the curing process, or through at least 95% of the curing process. After curing, the tube mandrel can be removed using a screw-like mechanism (e.g., the removal system 100 shown in FIG. 10), a hydraulic mechanism or a level mechanism, for example, to pull the tube mandrel free (e.g., out) of the latex glove. The latex glove can then be stripped from the hand mandrel (e.g., removed from the hand mandrel). The levers, gears, and other components can reset in order to be ready for the next manufacturing cycle (e.g., the next dipping round, the next batch).

In various other embodiments, the mandrel assembly 50 may allow the hand mandrel 2 and the tube mandrel 6 to be in close proximity to one another, without moving parts. After one round of integral dipping, the combined unit may be stripped from the hand mandrel 2 and the tube mandrel 6. Or, in other embodiments, the unit may be blown off using air, liquid, or gaseous element to produce the combined unit as one piece from both the hand mandrel 2 and the tube mandrel 6 at the same or similar time.

Some embodiments include a porcelain hand mandrel and/or a metal hand mandrel. The tube mandrel can be straight and/or curved. The tube mandrel can be a long metal bar that is smooth at the end that is dipped in the latex. The other end of the tube mandrel can have slots in at least one side. A screw mechanism, hydraulic system, or lever mechanism may engage the tube mandrel and can pull the tube mandrel out of the glove (e.g., a finished latex glove).

Gears can turn the hand mandrel and a brush to create a bead cuff on the glove. The tube mandrel can turn to keep the latex evenly distributed. Once at least a full rotation of the hand mandrel is completed to form a cuff, the hand mandrel and the tube mandrel can be brought into proximity. At this point, the hand mandrel and the tube mandrel can be locked into place (relative to each other) and can start to turn as one unit.

On a conveyor belt, the hand mandrel and the tube mandrel can be locked together and can go through a curing process together. After the curing is done, the tube mandrel can be withdrawn from the glove (e.g., through the use of a screw mechanism on the side of the tube mandrel to pull the tube mandrel out of the latex glove). Once the tube mandrel has been removed, the rest of the finished latex glove can be stripped from the hand mandrel. After glove removal, the gears and levers can be set back to a starting position (to enable the system to manufacture another glove).

In several embodiments, the hand mandrel and the tube mandrel can be separated by at least 2 inches at the narrowest part (e.g., at the narrowest section) when they are at least partially immersed in latex. This immersion can take place while the hand mandrel and the tube mandrel are oriented vertically (as shown in FIG. 2).

After removing the hand mandrel and the tube mandrel from the latex dip, the hand mandrel and the tube mandrel can be oriented horizontally. While the latex on the mandrels is in gel form (and/or in an uncured state) on the hand mandrel and on the tube mandrel, the hand mandrel (e.g., with a cylindrically shaped cuff) and the tube mandrel can be rotated (e.g., while they are horizontal). A brush can be used to form a cuff on the proximal end of the glove (e.g., in a wrist portion). The brush can include a cylindrical cotton wheel. While the hand mandrel is rotated to form the cuff, the tube mandrel can be rotated (e.g., separately, but at the same rotational speed as the hand mandrel). The tube mandrel can be kept away from the hand mandrel such that the tube mandrel does not get in the way of the hand mandrel (e.g., does not impede the cuff formation process).

After the formation of the cuff, the tube mandrel can be locked (or fixed) in position relative to the hand mandrel such that the tube mandrel can be rotated with the hand mandrel (e.g., as one unit). In this locked (or fixed) position, material that coats the hand mandrel can touch material that coats the tube mandrel. The material that coats the hand mandrel and the material that coats the tube mandrel can then be subjected to a vulcanization process.

After the vulcanization process, the tube mandrel can be withdrawn from the glove. The glove can then be removed (e.g., stripped) from the hand mandrel. The mandrels can be coated with a releasing agent to facilitate removal (e.g., stripping).

After the glove has been removed from the tube mandrel and the hand mandrel, the hand mandrel and the tube mandrel can be prepared for manufacturing another glove. Some embodiments use a batch process to manufacture multiple gloves in one cycle (e.g., at once).

In various embodiments, once the tube and glove have been removed from their respective tube and glove mandrels (or created by other means such as being sewn), the tube and glove may be attached to each other using various methods known to those skilled in the art, including, but not limited to, taping, stapling, sewing, gluing, VELCRO®, epoxy, strapping methods, and/or light or radiowave curing. For example, in one embodiment, VELCRO® may attach to one side of the glove (which may be, for example latex dipped or sewn); and the opposite VELCRO® piece may be attached to the tube.

In various embodiments of a sewn, cloth, or leather glove with a tube assembly, the assembly may be used in various industries and/or mechanical applications. For example, in the airline industry, a glove/tube unit may be used by mechanics to inspect airplanes with fiber optics carried through the tube assembly. In other embodiments for environmental sampling, the tube may contain, for example, pH, specific gravity, temperature, density, or metric sensors/detectors that allow such sampling for various industries using gloves.

Although gloves, glove manufacturing methods, and glove manufacturing assemblies have been disclosed in the context of certain embodiments and examples, it will be understood by those skilled in the art that the gloves, methods, and assemblies extend beyond the specifically disclosed embodiments to other alternative embodiments and/or uses of the embodiments and certain modifications and equivalents thereof. In certain embodiments various components are integrated and/or replaced by a single component. It should be understood that various features and aspects of the disclosed embodiments can be combined with or substituted for one another in order to form varying modes of glove systems. Accordingly, it is intended that the scope of the systems and methods herein-disclosed should not be limited by the particular disclosed embodiments described above, but should be determined only by a fair reading of current and/or future claims.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to a value, amount, or characteristic that is within less than 10% of, within less than 5% of, within less than 1% of, within less than 0.1% of, and within less than 0.01% of the stated value, amount, or characteristic.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A; some embodiments can include B; some embodiments can include C; some embodiments can include A and B; some embodiments can include A and C; some embodiments can include B and C; and some embodiments include A, B, and C. The term “and/or” is used to avoid unnecessary redundancy.

The various features and processes described above may be used independently of one another, or may be combined in various ways. All possible combinations and subcombinations are intended to fall within the scope of this disclosure. In addition, certain method, event, state, or process blocks may be omitted in some implementations. The methods and processes described herein are also not limited to any particular sequence, and the blocks or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

What is claimed is:

1. A method comprising:
rotating a first gear, wherein the rotating the first gear further rotates a first shaft coupled to a glove mandrel with a groove shaped along a finger of the glove mandrel;
rotating a second gear, wherein the rotating the second gear further rotates a second shaft coupled to a tube mandrel shaped to match the groove of the glove mandrel;
rotating a third shaft, wherein the third shaft is coupled to a base, wherein the base is coupled to the first gear and the second gear; and
dipping the glove mandrel and the tube mandrel into a dipping container, wherein the dipping container contains material capable of adhering to the glove mandrel and the tube mandrel.

2. The method of claim 1 further comprising:
activating a first lever coupled to the base to cause the rotating of the first gear and the rotating of the second gear;
sliding the rotating first gear in a first slot within the base;
sliding the rotating second gear in a second slot within the base; and
raising the dipping container to immerse the glove mandrel and the tube mandrel.

3. The method of claim 1 further comprising:
curing a tube formed around the tube mandrel and a glove formed around the glove mandrel;
stripping the tube mandrel and the glove mandrel; and
lining the tube formed around the tube mandrel with one or more optical fibers.

4. The method of claim 3, wherein the stripping the tube mandrel and the glove mandrel comprises:
activating a second lever to start a motor;
the motor causing a wheel with gear teeth to rotate;
rotating the wheel against the tube mandrel;
causing, by the rotating of the wheel, the tube mandrel to move along the rotating wheel;
removing the tube from the tube mandrel; and
stripping the glove from the glove mandrel.

5. The method of claim 3, wherein the curing the tube further comprises:
halting the rotating of the first gear;
halting the rotating of the second gear;
sliding the first gear and the second gear within the first slot and second slot so that the glove mandrel and the tube mandrel are near each other; and
curing the tube formed around the tube mandrel within the groove shaped along the finger of the glove mandrel.

6. The method of claim 1 further comprising:
blowing off, with gas pressure, a glove from the glove mandrel and a tube from the tube mandrel; and
lining the interior of a tube formed around the tube mandrel with one or more sensory devices.

7. The method of claim 6, wherein the one or more sensory devices includes camera and video devices.

8. The method of claim 6, wherein the tube mandrel is withdrawn using hydraulic or level mechanisms.

9. A system comprising:
a shaft;
a base connected to the shaft, wherein the shaft is operable to the move the base;
a hand mandrel with one or more grooves, coupled to the base; and
one or more tube mandrels coupled to the base, wherein at least one of the one or more tube mandrels corresponds to at least one of the one or more grooves.

10. The system of claim 9, wherein the one or more grooves of the hand mandrel are situated along a finger of the hand mandrel,
wherein the hand mandrel and the at least one tube mandrel are located in close proximity to each other to allow a tube, formed around the at least one tube mandrel, to be directly cured within one of the one or more grooves.

11. The system of claim 9 further comprising:
a gas pressure component coupled to the hand mandrel and the one or more tube mandrels, wherein the hand mandrel and the one or more tube mandrels contain one or more holes operable to allow a gas flow; and
a switch operable to activate the gas pressure component, wherein the gas pressure component is operable to contemporaneously blow through the one or more holes, with the gas flow, a glove cured around the hand mandrel and one or more tubes cured around the one or more tube mandrels.

12. The system of claim 11, wherein a gas pressure component on the exterior of the latex, nitrile, neoprene dipped glove once cured is blown between the mandrel and the formed glove at the base/wrist, blowing off the finished product.

13. The system of claim 9 further comprising:
a first gear coupling the hand mandrel to the base;
a second gear coupling the one or more tube mandrels to the base; and
a first lever operable to activate the first gear and second gear and to activate a lowering of the system into a container.

14. The system of claim 13 further comprising:
a second lever operable to start a motor;
a hydraulic mechanism, a lever mechanism or a wheel with indentations driven by the motor and operable to move proximally to the one or more tube mandrels and one or more circular rings with empty centers allowing the one or more tube mandrels to be stripped by the wheel with indentations.

15. A system comprising:
a glove with one or more digits; and
one or more tubes, coupled to the exterior of at least one of the one or more digits.

16. The system of claim 15, wherein the glove is a leather glove and one or more tubes are latex tubes, wherein the leather glove and latex tubes are connected via sewing, taping, gluing strapping or the attachment of a first VELCRO® strip on the leather glove and a second, opposing VELCRO® strip on the latex tube.

17. The system of claim 15, wherein the one or more tubes are latex tubes cured, via an integrated latex dipping process, to the exterior of a pointer digit of the one or more digits.

18. The system of claim 15, wherein the one or more tubes carry endoscopic fibers.

19. The system of claim 15, wherein the one or more tubes contain optical fibers operable to act as a light source.

20. The system of claim 15, wherein a first tube of the one or more tubes contains a pH meter or other sensing device, wherein a second tube of the one or more tubes contains at least one optical fiber.

21. The system of claim 15, wherein the glove has been formed with a hand mandrel, wherein the one or more tubes have been formed with one or more tube mandrels, wherein the hand mandrel and the one or more tube mandrels are part of a system operable for latex formation.

22. The system of claim 21, wherein the glove and the one or more tubes have been formed contemporaneously in the system operable for latex formation.
